(19) United States
(12) Patent Application Publication
(10) Pub. No.: US 2023/0191051 A1
(43) Pub. Date: Jun. 22, 2023

(54) FILTER ARRANGEMENT AND PROCESS FOR FILTERING A GAS FROM A GAS MIXTURE

(71) Applicant: Drägerwerk AG & Co. KGaA, Lübeck (DE)

(72) Inventors: Dominik TRITSCHER, Lübeck (DE); Marco MONNIG, Lübeck (DE); Jonas Gregor SCHMID, Lübeck (DE)

(21) Appl. No.: 18/065,714

(22) Filed: Dec. 14, 2022

(30) Foreign Application Priority Data
Dec. 16, 2021 (DE) 10 2021 133 423.3

(51) Int. Cl. A61M 16/00; B01D 53/18

(52) U.S. Cl. CPC A61M 16/0093; A61M 16/0003; B01D 53/18; A61M 2205/3368; B01D 2201/56; B01D 2259/4533

(57) ABSTRACT

An arrangement and a process filter out at least one gas from a gas mixture. A filter unit (4) of the filter arrangement comprises an inlet and an outlet and is adapted to filter the gas out of the gas mixture while the gas mixture flows through the filter unit (4). The filter unit (4) takes up the gas and heats up in the process. A filter temperature sensor (46, 46.2) of the filter arrangement is adapted to measure at least once an indicator of the temperature in a first measuring area (MP, MP.2) inside the filter unit (4). Depending on the measured temperature, a message is generated and output in a form perceptible by a human being. This message includes information about the current state of the filter unit (4).

### CROSS REFERENCE TO RELATED APPLICATIONS

This application claims the benefit of priority under 35 U.S.C. § 119 of German Application 10 2021 133 423.3, filed Dec. 16, 2021, the entire contents of which are incorporated herein by reference.

### TECHNICAL FIELD

The invention relates to a filter arrangement and a process for filtering out at least one gas from a gas mixture.

### BACKGROUND

The task of filtering a gas out of a gas mixture occurs, for example, in a hospital. A patient is artificially ventilated and is or has been sedated or anesthetized with at least one anesthetic (anesthetic). In artificial ventilation, an anesthesia machine performs a sequence of ventilation strokes and delivers an amount of a gas mixture comprising oxygen and anesthetic to the patient in each ventilation stroke. The breathing air that the patient exhales therefore usually contains traces of this anesthetic.

One aim is to prevent exhaled anesthetic from entering the environment of the anesthesia machine. A ventilation circuit is therefore established to return exhaled air to the anesthesia machine. The air exhaled by the patient is returned to the anesthesia machine.

In this ventilation circuit between the patient and the anesthesia machine, excess gas is typically generated and must be removed from the ventilation circuit. In one embodiment, the excess gas is supplied to a stationary fluid collection system. The purpose is to prevent exhaled anesthetic as part of the excess gas from entering the fluid intake, and thereby potentially entering a hospital supply system.

In this application, the excess gas functions as the gas mixture, and the anesthetic agent or each anesthetic agent (the anesthetic) in the gas mixture functions as the gas or a gas to be filtered out. It is possible that the excess gas contains at least two different anesthetic agents (an anesthetic agent and two or more anesthetic agents are also referred to herein as anesthetic), all of which are to be filtered out.

The procedure of passing the gas mixture, in this case the excess gas, through a filter unit is known. The filter unit filters the gas to be removed from the gas mixture, in this case the anesthetic, out of the gas mixture while the gas mixture flows through the filter unit. Inevitably, the filter unit absorbs the anesthetic filtered out in this process and can therefore only filter out a certain amount of anesthetic. Therefore, it is necessary from time to time to replace a used filter unit with a new filter unit.

US 2001 / 0 025 640 A1 proposes detecting anesthetic in a gas mixture by using an indicator material, this indicator material reacting chemically with the anesthetic and changing its color as a result of the reaction. A user may visually perceive a color change and then replace a filter unit, cf. par. [0015].

In FIG. 1 of WO 2019 / 038 566 A1, a ventilation circuit is described in which a patient is supplied with a gas mixture comprising anesthetic. The gas mixture exhaled by the patient (waste gas 38) may comprise anesthetics and is passed through a filter unit comprising a housing (canister 208) and a filter material 210. The housing 208 includes an inlet 203 and an outlet 211, 311, and the filter material 210 binds anesthetic. Ambient air (general theatre air 206) and cooled air (cold air stream 204a) are also passed into the filter unit 208, 210 through an inlet 204. This cools the stream of gas exiting the filter unit 208, 210. A controller 301 receives a signal from a sensor (thermistor or thermocouple 303) in the outlet 311 of the housing 208 and controls the supply of cold air through the inlet 204. If anesthetic is detected in the outlet 211, the gas mixture is passed through a filter 214 containing activated carbon.

### SUMMARY

It is the object of the invention to provide a filter arrangement and a process which, by means of a filter unit, filter out at least one gas from a gas mixture while the gas mixture is flowing through the filter unit, and which prevent, in many cases, with greater reliability than known filter arrangements and processes, the undesired event of gas to be filtered out being present downstream of the filter unit.

The invention is solved by a filter arrangement having the features according to the invention and by a process having the features according to the invention. Advantageous embodiments are indicated herein. Advantageous embodiments of the filter arrangement according to the invention are, as far as useful, also advantageous embodiments of the process according to the invention and vice versa.

The filter arrangement according to the invention and the process according to the invention are capable of filtering out at least one gas from a gas mixture. The gas mixture is, for example, the breathing air exhaled by a patient or excess air in a ventilation circuit, and the gas to be filtered out is anesthetic.

The filter arrangement comprises a filter unit having an inlet and an outlet. The filter arrangement is configured as follows: The gas mixture flows into the filter unit through the inlet, flows through the filter unit at least once, optionally multiple times, and flows through the outlet out of the filter unit. The filter unit is adapted to filter the gas from the gas mixture while the gas mixture is flowing through the filter unit.

The filter unit is able to take up (to absorb) the filtered gas. As a result of the process of taking up the filtered gas, the filter unit heats up.

The filter arrangement further comprises a sensor arrangement comprising a first filter temperature sensor and optionally comprising at least one second filter temperature sensor. The first filter temperature sensor is configured to measure at least once an indicator of a temperature inside the filter unit, namely the temperature in a first measuring area. Preferably, an indicator of the instantaneous temperature in the first measuring area is measured. The second filter temperature sensor or each second filter temperature sensor is also configured to measure at least once an indicator of a temperature inside the filter unit, preferably in each case in a respective second measuring area which is spatially separated from the first measuring area and is particularly preferably arranged upstream of the first measuring area.

The filter arrangement according to the invention is configured to automatically perform the following steps:
The filter arrangement generates a message depending on the measured temperature in the first measuring area - more precisely: depending on at least one measured value of the temperature. This message comprises information about the current state of the filter unit. The information comprises, for example, the message that the filter unit should be replaced as soon as possible, or a prediction of how long the filter unit can still be used, or also that the filter unit does not currently need to be replaced.
In one alternative, the filter arrangement outputs the message with this information in a form that can be perceived by a human being, in particular visually, acoustically and/or haptically (by vibrations), for example on a wall of a housing of the filter unit that is visible from the outside. In another alternative, the filter arrangement causes this message to be output in a form that can be perceived by a human being, by a spatially remote receiver. Preferably, this receiver is at least temporarily in a wired or wireless data connection with the filter arrangement.

The process according to the invention is carried out using a filter arrangement according to the invention. The process comprises the following steps:
The gas mixture flows through the inlet into the filter unit. The gas mixture flows through the filter unit at least once, in one embodiment several times.
The filter unit filters the gas - or at least a part of the gas - out of the gas mixture while the gas mixture flows through the filter unit.
The gas mixture then flows through the outlet out of the filter unit.
The filter unit takes up (absorbs) the gas which the filter unit has filtered out of the gas mixture flowing therethrough. The filter unit is configured as follows: As a result of the process of taking up the gas, the filter unit heats up.
The first filter temperature sensor measures at least once the indicator for the temperature in the first measuring area.
Depending on at least one measured value of the temperature in the first measuring area, a message is automatically generated. This message includes information about the current state of the filter unit.
This message is set to be output in a form that can be perceived by a human being.

According to the invention, a filter unit is used which is capable of taking up (absorbing) the gas to be filtered out and heats up in the process of filtering out. Preferably, an exothermic chemical reaction takes place in the filter unit. Many known filter materials heat up when they take up (absorb) a gas, such as an anesthetic or other long-chain hydrocarbons. An example is a filter material comprising activated carbon. In one embodiment, the filter unit comprises a cartridge and a bulk material inside the cartridge, this bulk material comprising activated carbon or other absorbent material. In one embodiment, it is possible to reuse the cartridge and replace only the bulk material. In another embodiment, the cartridge with the bulk material can be replaced only as a whole.

The invention provides that heating of the filter unit during a use is used to determine a current state of the filter unit and to cause a user to be informed of that current state. Because a temperature is measured inside the filter unit, it is possible, but thanks to the invention in many embodiments not necessary, for a sensor to come into contact with a filter material inside the filter unit. In particular, it is not necessary for a sensor to chemically react with the filter material and/or chemically determine what amount of gas the filter material has absorbed to date. Rather, in many embodiments, the invention makes it possible to determine the temperature and thus the current state of the filter unit without contact from the outside. This effect facilitates in many cases a monitoring of the filter unit, including remote monitoring. Furthermore, if the temperature inside the filter unit is measured without contact, there is less risk that a gap in the filter unit will cause the gas mixture or bulk material to escape.

In many embodiments, the invention also eliminates the need to place a sensor inside the filter unit. Often, such a sensor would have to be discarded or disposed of along with the filter unit when the filter unit is used up and replaced with a new filter unit. In many embodiments, however, the invention allows the same filter temperature sensor to be reused sequentially for multiple filter units.

The first filter temperature sensor and optionally at least one second filter temperature sensor each measure, at least once, an indicator of a temperature occurring in a respective measuring area inside the filter unit. This temperature in the measuring area inside the filter unit is an indicator of how much gas to be filtered out the filter unit has taken up so far in this measuring area.

Generally, the filter unit does not absorb the gas to be filtered out uniformly over the entire extent of the filter unit. Rather, at any one time, usually only one area inside the filter unit filters out the gas, absorbs the gas, and heats up during this process. When this area can no longer take up (absorb) any more gas, the gas mixture flows through this area without being absorbed, and the gas is only filtered out in an area downstream. Thus, an absorption region is formed in the filter unit, which is the region that currently absorbs gas. This absorption region thus migrates through the filter unit from the inlet in the direction of flow of the gas mixture to the outlet. The invention makes use of the fact that the absorption of the gas leads to heating and that often the absorption area, and thus an area of increased temperature, migrates through the filter unit.

According to the invention, the gas mixture flows at least once from the inlet through the filter unit to the outlet. The first measuring area, the temperature of which the first filter temperature sensor measures at least once, can be positioned close to the outlet of the filter unit on the one hand. Thus, if the above-mentioned absorption region has reached this first measuring area near the outlet, the filter unit is only able to absorb a small amount of further gas because there is only a small amount of filter material downstream of the first measuring area and upstream of the outlet. The event that this absorption area (first measuring area) near the outlet heats up is detected. This event is an indication that the filter unit should be replaced.

Positioning the first measuring area close to the outlet also ensures in many cases that the filter unit is only replaced when it actually needs to be replaced, and not significantly too early. This positioning of the first measuring area close to the outlet thus reduces the consumption of filter units compared to a positioning of the first measuring area further upstream. On the other hand, the first measuring area can be positioned with a sufficiently large safety distance to the outlet. This makes it possible to reduce the risk that a relevant amount of the gas to be filtered out leaves the filter unit through the outlet, because the filter unit is replaced too late. In addition, the safety distance makes it possible in many cases to still have sufficient time to replace the filter unit after an appropriate message has been issued.

In many cases, the invention enables the following: A message that the filter unit must now be replaced can be generated in due time, but not substantially too early, and output in a form that can be perceived by a human being. The filter unit can then be replaced in due time without, for example, endangering ongoing medical treatment of an anesthetized patient.

The invention makes it possible to replace the filter unit depending on the amount of gas actually absorbed, i.e. event-based. The invention avoids the need to replace the filter unit on a time basis, i.e. at regular intervals and irrespective of how much gas the filter unit has actually absorbed so far.

The invention can be used in combination with a sensor capable of detecting, at a measuring position downstream of the filter unit, whether or not the gas mixture exiting the filter unit still contains the gas to be filtered out. This sensor, located downstream of the filter unit, detects, for example, an anesthetic in the escaping gas mixture. However, the invention avoids the need to provide such a sensor and to replace the filter unit only when this sensor actually detects the gas to be filtered out downstream of the filter unit. Such a sensor is only able to detect the undesired event that a so-called filter breakthrough has occurred. In the case of a filter breakthrough, the filter unit is no longer able to filter the gas completely out of the gas mixture. After a filter breakthrough, a relevant amount of the gas often escapes from the outlet and then often enters the environment or a fluid intake. A sensor with a measuring area downstream of the filter unit is able to detect such a filter breakthrough, but in many cases too late to prevent a filter breakthrough.

Moreover, particularly during medical treatment of an anesthetized patient, it is sometimes not possible to replace the filter unit immediately upon discovery of a filter breakthrough. The invention reduces the risk of this undesirable event occurring. In many cases, the invention makes it possible to replace the filter unit in a timely manner without jeopardizing the medical treatment.

The invention can be used in combination with a quantity sensor, which determines what quantity of the gas to be filtered out has flowed into the filter unit through the inlet as a component of the gas mixture and has been absorbed by the filter unit since the time when the use of the filter unit was started. However, the invention avoids the need to replace the filter unit depending on results from such a quantity sensor. In many cases, such a quantity sensor is only able to measure the quantity which the filter unit has absorbed so far relatively unreliably.

The invention makes it possible to detect in a relatively simple manner when the filter unit needs to be replaced, and in many cases before a filter breakthrough has occurred. Relatively simple and reliable temperature sensors are available on the market, which in some embodiments can also be used for the filter arrangement according to the invention.

According to the invention, the first filter temperature sensor measures an indicator of the current temperature in the first measuring area inside the filter unit. The measured temperature in the first measuring area is used for generating the message, and can be used for deciding whether the filter unit can be further used or is used up. In one embodiment, the generation of this message is based on the measured temperature in the first measuring area and optionally on the measured temperature in a second measuring area.

In one embodiment, the filter arrangement according to the invention further comprises a signal processing evaluation unit. The evaluation unit may be arranged spatially remote from the filter unit. The evaluation unit receives a signal from each of the first and at least one optional second filter temperature sensor. Using this signal or these signals, the evaluation unit automatically decides whether a predetermined criterion is met or not. This criterion depends on at least one value of the temperature in the first measuring area, optionally additionally on at least one value of the temperature in the second measuring area. If this criterion is fulfilled, the evaluation unit generates the message with the information about the current state of the filter unit. The evaluation unit causes this message to be output in a form that can be perceived by a human, preferably by a spatially remote receiver. Preferably, this message comprises information that the filter unit is used up or will soon be used up and therefore needs to be replaced. The message may additionally comprise a prediction of how long the filter unit can still be used until it needs to be replaced.

Preferably, the process according to the invention comprises the following steps: A decision is automatically made as to whether the predetermined criterion has been met. If the criterion is met, the message is generated with the information about the current state of the filter unit.

According to the invention, the first filter temperature sensor measures the temperature in the first measuring area at least once. In a preferred embodiment, the first filter temperature sensor measures the temperature in the first measuring area several times in succession while the gas mixture is flowing through the filter unit, namely at several successive sampling times. Using a signal from the first filter temperature sensor, the signal processing evaluation unit determines a time course of the measured temperature in the first measuring area. Of course, the evaluation unit is only able to determine this time course approximately. According to the invention, the evaluation unit generates a message when the predetermined criterion is met. According to the preferred embodiment just described, this criterion depends on the determined time course of the temperature in the first measuring area.

The filter unit heats up in the first measuring area while it absorbs the gas to be filtered out in the first measuring area. In many cases the arrangement that the time course of the temperature is determined makes it possible to make a prediction with higher reliability as to when the filter unit is no longer able to absorb any further gas in the first measuring area, compared to a prediction based only on one measured value. The absorption area mentioned above has then migrated through the first measuring area and further towards the outlet. It is often possible for the evaluation unit to make this prediction automatically. This prediction makes it possible to replace the filter unit in due time with even greater certainty. In due time means: before a filter breakthrough, i.e. before the gas to be filtered out escapes from the outlet of the filter unit. Furthermore, the embodiment with the time course makes it possible in some cases to distinguish with even greater certainty the process that the filter unit absorbs gas and thereby heats up from the process that the filter unit heats up due to a sufficiently large or increased ambient temperature or any other external influence.

According to the invention, the first filter temperature sensor measures at least once an indicator of temperature in the first measuring area inside the filter unit. In a preferred embodiment, the sensor arrangement comprises at least one second filter temperature sensor. The second filter temperature sensor or each second filter temperature sensor is also capable of measuring an indicator of the temperature inside the filter unit, preferably the same indicator as the first filter temperature sensor. However, the two filter temperature sensors may also measure different indicators of temperature. The first filter temperature sensor is capable of measuring the indicator of the temperature at the first measuring area, and the second filter temperature sensor or each second filter temperature sensor is capable of measuring the indicator of the temperature at a respective second measuring area inside the filter unit. Viewed in the direction of flow in which the gas mixture flows through the filter unit, the first measuring area is located downstream of the second measuring area or each second measuring area. Thus, the first measuring area is located between the or each second measuring area and the outlet of the filter unit. It is possible that a plurality of second measuring areas are arranged spatially separated apart from each other between the inlet and the first measuring area.

The embodiment with multiple filter temperature sensors can be combined with the embodiment that the filter arrangement has an evaluation unit. The embodiment with several filter temperature sensors can also be implemented without an evaluation unit.

The evaluation unit preferably determines at least once a spatial course of the temperature along a path leading from the inlet to the outlet of the filter unit. This spatial course relates to a point in time. In order to determine this spatial course, the evaluation unit uses the signal of the first filter temperature sensor and the respective signal of the or at least one second filter temperature sensor. According to the invention, the evaluation unit generates the message when the predetermined criterion is met. According to the embodiment just described, the criterion depends on the spatial course of the temperature along the path. Preferably, the evaluation unit determines the current spatial course several times in succession.

The embodiment with multiple filter temperature sensors creates redundancy and allows the filter arrangement according to the invention to further be used even if a filter temperature sensor has failed. It is possible to use sensors that apply different measurement principles for determining the filter temperature. This also increases reliability.

While the gas mixture flows through the filter unit and the filter unit absorbs the gas to be filtered out, the filter unit often does not heat uniformly over its entire extent. Rather, at any given time an absorption area inside the filter unit is heating up, and this absorption area migrates over time through the filter unit from the inlet to the outlet until the filter unit can no longer absorb any more gas. The embodiment that the first measuring area is arranged downstream of the second measuring area makes it possible to exploit this fact just described. In general, first the second filter temperature sensor measures an elevated temperature and afterwards the first filter temperature sensor measures an elevated temperature. In many cases, the embodiment with two different measuring areas inside the filter unit makes it possible to predict with even higher reliability when the filter unit is or will be used up. This advantage is achieved in particular if the evaluation unit determines at least once a spatial course of the filter temperature.

It is possible that the sensor arrangement comprises a third and optionally a fourth filter temperature sensor, wherein these further sensors measure the indicator or each indicator of temperature in a third or an optional fourth measuring area inside the filter unit. Viewed in the direction of flow of the gas mixture through the filter unit, the first measuring area is arranged downstream of the second measuring area, the second measuring area is arranged downstream of the third measuring area, and the third measuring area is arranged downstream of the optional fourth measuring area.

In a preferred embodiment, the decision about the current state of the filter unit is additionally based on the ambient temperature, for example the temperature in a room where the filter arrangement is used. This is because, as a rule, the temperature inside the filter unit depends not only on the amount of gas absorbed, but additionally on the ambient temperature. The aforementioned signal-processing evaluation unit of the sensor arrangement compares at least one measured value for the temperature in the first measuring area with a measured ambient temperature. Depending on the result of the comparison, the evaluation unit automatically generates the message with information about the current state of the filter unit. The given criterion thus depends on the difference between the temperature in the first measuring area and the ambient temperature. This message is output in a form perceptible by a human being. Optionally, the evaluation unit compares the variation over time of the temperature in the first measuring area with the ambient temperature. In many cases, the ambient temperature can be considered to be constant over time and / or over space. It is also possible to measure the ambient temperature several times in succession. In one embodiment, the evaluation unit compares a spatial variation of the filter temperature with the ambient temperature at least once, with the spatial variation and the ambient temperature preferably relating to the same point in time.

In one implementation of this embodiment, the sensor arrangement comprises an ambient temperature sensor. This ambient temperature sensor is adapted to measure an indicator of temperature in the environment of the filter arrangement. In another implementation, the sensor arrangement is adapted to receive a signal, this signal comprising an indicator of ambient temperature. This indicator of ambient temperature was measured by an external ambient temperature sensor. “External” means that the ambient temperature sensor is positioned spatially remote from the filter arrangement and is not necessarily a component of the filter arrangement.

In some cases, the embodiment in which the ambient temperature is measured and used makes it possible to more quickly detect the event that the filter unit is about to be replaced. Indeed, in some cases, it is possible to detect this event without determining a temporal or spatial course of the temperature.

Several possible embodiments of the first filter temperature sensor are described below. The optional second and the optional third filter temperature sensor may also be configured according to a respective one of these embodiments. The embodiments may also be combined, i.e. the first filter temperature sensor is implemented according to a first embodiment and the second filter temperature sensor is implemented according to a different second embodiment. It is also possible that at least two filter temperature sensors of the filter arrangement are implemented according to the same embodiment.

In a preferred embodiment, the filter unit comprises a filter mount and a filter. During operation, the filter is inserted into the filter mount. The filter comprises a filter material capable of filtering out and taking up (absorbing) the gas wherein the filter heats up when taking up the gas. The gas mixture flows into the filter mount through an inlet opening, into the filter through the inlet, through the filter at least once, through the outlet out of the filter, and out of the filter mount through an outlet opening. Preferably, when the filter is in place, the inlet and outlet of the filter are within the space enclosed by the filter mount. The filter can be removed from the filter mount and replaced with a new filter. Preferably, the same filter mount accommodates several filters, one after the other.

Typically, the rest of the filter arrangement remains unchanged when a filter is replaced. In particular, it is not necessary to interrupt a fluid connection between the filter mount and a medical device or fluid receptacle to replace the filter. The gas mixture containing the gas to be filtered out is guided to the filter mount, then flows through the filter and back out of the filter mount with this filter inserted into the filter mount.

In one embodiment, the first filter temperature sensor is inserted into a wall of the filter mount. A distance occurs between the first filter temperature sensor in the wall and the filter inserted in the filter mount. The first filter temperature sensor is therefore also at spatial distance from the first measuring area.

This embodiment eliminates the need to provide the filter itself with the filter temperature sensor. Instead, the same filter temperature sensor in the wall of the filter mount can be used successively for several inserted filters. This reduces the amount of material required. Furthermore, it is easier to establish a wired data connection between the first filter temperature sensor in the wall and a signal processing evaluation unit than if the filter temperature sensor were part of the inserted filter.

A distance occurs between the first filter temperature sensor in the wall of the filter mount and the first measuring area. The inserted filter heats up, also in the first measuring area, while the filter takes up (absorbs) the gas to be filtered out from the gas mixture flowing through. The first measuring area therefore emits a greater amount of electromagnetic radiation in the infrared range as it picks up the gas and therefore heats up, compared to another condition.

In one embodiment, the first filter temperature sensor in the wall of the filter mount is configured as an infrared sensor or comprises at least one infrared sensor. The infrared sensor or each infrared sensor is capable of measuring an indicator of the amount and/or intensity of infrared radiation emitted by the inserted filter and impinging on the infrared sensor. Other implementations of a sensor in the wall that measures the filter temperature in a non-contact manner are also possible.

In many cases, the embodiment with the filter temperature sensor in the wall eliminates the need for the actual dimensions of the filter and/or the filter mount to exactly match predetermined dimensions and for the filter to be correctly positioned in the filter mount. Rather, in many cases the infrared sensor is able to measure the temperature in the first measuring area sufficiently reliably even if the distance between the first measuring area and the infrared sensor varies from filter to filter.

In another embodiment of the first filter temperature sensor in the wall of the filter mount, a thermal sensing element (a thermal contact surface) provides thermal contact between the filter and the first filter temperature sensor. The thermal contact extends into the first sensing area. The thermal contact bridges the distance between the filter and the filter mount and conducts a heating of the filter to the first filter temperature sensor. The first filter temperature sensor further comprises a transducer. This transducer is adapted to generate a signal, preferably an electrical signal, depending on the actual temperature of the sensing element, depending on a temperature of the thermal contact.

The configuration with the thermal contact leads in some cases to a particularly simple mechanical embodiment and is in some cases more robust against environmental influences, for example contamination, than other possible embodiments.

In another embodiment, the first filter temperature sensor comprises a sensing element and a receiver. The sensing element is arranged inside the filter. The receiver is embedded into the wall of the filter mount. The sensing element is capable of generating a signal that depends on the temperature in the first measuring area. If the filter is inserted into the filter mount, a data link is established at least temporarily between the sensing element and the receiver, preferably a data link by radio waves. Via this data link the signal of the sensing element is transmitted to the receiver.

The configuration with the sensing element in the filter makes it possible to position the sensing element in the first measuring area or at least particularly close to the first measuring area. In some cases, even a relatively small temperature increase of the filter can be measured reliably and/or quickly. In some cases, the embodiment with the sensing element in the filter is less dependent on ambient conditions, in particular ambient temperature and ambient humidity.

In many cases, a further embodiment saves the need to acquire and process electrical measured values or electrical signals. Therefore, this further embodiment generally obviates the need to use a signal-processing evaluation unit for the filter arrangement. It is also possible to use the embodiment described below in conjunction with an evaluation unit.

According to this further embodiment, the first filter temperature sensor comprises a chemical indicator element. This chemical indicator element is in thermal contact with the filter unit. For example, the chemical indicator element is externally applied to a filter of the filter unit. Preferably the chemical indicator element is mounted until the filter unit, preferably on a filter of the filter unit, such that the chemical indicator element is visible from outside. The chemical indicator element is either in a first state or in at least a second state, wherein whether the chemical indicator element is in the first state or in the second state or a second state depends on the temperature in the first measurement region. Whether the chemical indicator element is in the first state or the second state can be visually perceived. The two states differ from each other in a visually perceptible manner. For example, an increase in temperature in the first measurement state results in a change in color. Thus, the filter arrangement visually outputs the message with the information about the current state using the chemical indicator element.

In one embodiment, the embodiment with the chemical indicator element is combined with the embodiment that the filter unit comprises a filter and a filter mount wherein the filter is inserted or can be inserted into the filter mount. According to this combination the chemical indicator element is mounted onto the filter. The filter mount surrounds the filter with the chemical indicator element. Preferably, a viewing window is provided in the filter mount so that a user can determine the current state of the chemical indicator element from the outside through the viewing window. This embodiment leads to a particularly simple implementation that does not require a signal processing evaluation unit. In another embodiment, the sensor arrangement comprises a color sensor, for example a camera, wherein the color sensor can automatically determine the state of the chemical indicator element. The embodiment with the viewing window and the embodiment with the color sensor can be combined, for example by the color sensor detecting the state of the chemical indicator element through the viewing window.

In one embodiment, the chemical indicator element covers the entire circumference of the filter. For example, if the filter is in the form of a cylinder, the chemical indicator element is in the form of a strip on the circumferential surface. The embodiment in which the chemical indicator element covers the entire circumference of the filter eliminates the need to insert the filter into the filter mount in a particular rotational position. Rather, in any positioning of the inserted filter, the chemical indicator element is visible through the viewing window.

Possibly, the filter unit comprises at least two chemical indicator elements, one chemical indicator element being arranged downstream of the other chemical indicator element. The sequence of chemical indicator elements visually indicates how the absorption region described above migrates from the inlet through the filter unit to the outlet.

According to the invention, the filter unit is able to filter out at least one gas from a gas mixture. In one embodiment, the gas or each gas to be filtered out is an anesthetic, also called an anesthetic agent. Many anesthetics have a boiling point which is between 25° C. and 50° C., and therefore often evaporate at room temperature.

In one application, the filter arrangement according to the invention is used for artificial ventilation of a patient. The filter arrangement filters out a gas, for example an anesthetic or carbon dioxide, from a gas mixture which is directed towards the patient or directed away from the patient.

The invention further relates to a system capable of artificially ventilating a patient. This system includes a ventilator, a fluid guide unit with an inspiration portion, and a filter arrangement according to the invention. A tube, such as a two-lumen tube, and a tube are two examples of a fluid guide unit. For example, the patient-side coupling unit includes a breathing mask or a tube or a catheter.

The fluid guide unit at least temporarily provides fluid communication between the ventilator and a patient-side coupling unit. The patient-side coupling unit is positioned or positionable in or on the patient’s body. The ventilator is adapted to deliver a gas mixture through the inspiration portion, and thus through the established fluid connection, to the patient-side coupling unit. This gas mixture comprises oxygen and at least one further gas, for example breathing air and / or an anesthetic. The gas mixture reaches the patient’s respiratory system via the patient-side coupling unit. In one embodiment, the ventilator is capable of performing a sequence of ventilation strokes, wherein in each ventilation stroke a quantity of the gas mixture is delivered through the inspiration portion to the patient-side coupling unit.

The filter arrangement according to the invention is at least temporarily in fluid communication with the fluid guide unit between the ventilator and the patient-side coupling unit. The filter arrangement according to the invention filters out at least one gas from the gas mixture flowing through the fluid guide unit and through the filter unit of the filter arrangement. This gas is, for example, carbon dioxide or an anesthetic.

In a preferred embodiment, the ventilator is configured as an anesthesia machine (anesthesia device, anesthesia apparatus). The fluid guide unit comprises the inspiration portion and additionally an expiration portion. The fluid guide unit establishes or is configured to establish a ventilation circuit between the anesthesia machine and the patient-side coupling unit. Preferably, the ventilator configured as an anesthesia machine comprises an anesthetic vaporizer or anesthetic vaporizer configured to feed at least one gaseous anesthetic into a stream of carrier gas. The anesthesia machine delivers a gas mixture through the inspiration portion to the patient-side coupling unit, the gas mixture comprising oxygen and anesthetic and having been generated by the anesthetic vaporizer or anesthetic vaporizer. This gas mixture sedates or anesthetizes the patient. The filter arrangement according to the invention is at least temporarily in a fluid connection with the expiration portion. The filter arrangement is adapted to filter out the anesthetic from the gas mixture flowing through the expiration portion when the gas mixture is passed through the filter unit of the filter arrangement.

According to this embodiment, a ventilation circuit is established whereby the anesthesia machine delivers the gas mixture to the patient-side coupling unit and the air exhaled by the patient flows back through the expiration portion to the anesthesia machine. This exhaled air contains carbon dioxide and typically a part of the anesthetic supplied. Preferably, a CO2 absorber filters carbon dioxide from the exhaled air. Thanks to the ventilation circuit no exhaled air can reach in environment of the system.

In case the ventilation circuit just described is established, in one embodiment the filter arrangement according to the invention is arranged in that expiration portion of the ventilation circuit which conducts the exhaled air from the patient-side coupling unit back to the anesthesia machine. In one implementation the expiration portion comprises two segments wherein the filter arrangement is located between these two segments. The exhaled air is passed through the filter unit. In another embodiment, a quantity of the gas mixture is diverted from the ventilation circuit, preferably from the expiration portion, guided to the filter arrangement according to the invention, passed through the filter unit and guided back to the ventilation circuit.

The air exhaled by the patient usually contains anesthetic. Anesthetic and a carrier gas are usually added to the ventilation circuit, so that, conversely, an excess amount of the gas mixture must be removed from the ventilation circuit. The filter arrangement according to the invention reduces the risk of an anesthetic contained in the excess gas mixture leaking into the environment and affecting a person in the vicinity of the ventilation circuit or the stationary infrastructure of a hospital. It is also possible that the filter arrangement according to the invention filters carbon dioxide from the exhaled air. It is also possible that the filter arrangement according to the invention filters out at least one anesthetic agent (filters out anesthetic) as well as carbon dioxide.

In the following, the invention is described with reference to an example of an embodiment. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a ventilation system with an anesthesia machine and a filter unit;

FIG. 2 is a schematic side view showing an exemplary filter unit in which the inlet and the outlet of the filter are arranged near the bottom;

FIG. 3 is a schematic plan view showing another exemplary filter unit with a forced guidance of the excess gas;

FIG. 4 is a schematic view with graph showing an example of the spatial course of the temperature in the filter unit, i.e. the dependence of the temperature on the location;

FIG. 5 is a schematic sectional view showing a first embodiment of a filter temperature sensor comprising a sensing element within the filter element;

FIG. 6 is a schematic sectional view showing a second embodiment of a filter temperature sensor having a thermal contact element on the outer surface of the filter cartridge;

FIG. 7 is a schematic sectional view showing a third embodiment of a filter temperature sensor comprising a non-contact infrared sensor; and

FIG. 8 is a schematic sectional view showing a fourth embodiment of a filter temperature sensor having a plurality of chemical indicator elements on the filter cartridge.

### DESCRIPTION OF PREFERRED EMBODIMENTS

Referring to the drawings, in an embodiment, the invention is used to artificially ventilate a patient P while delivering at least one anesthetic agent (anesthetic) to the patient. The patient P is in an at least partially enclosed space while being artificially ventilated, for example in a room of a hospital or on board a vehicle or aircraft.

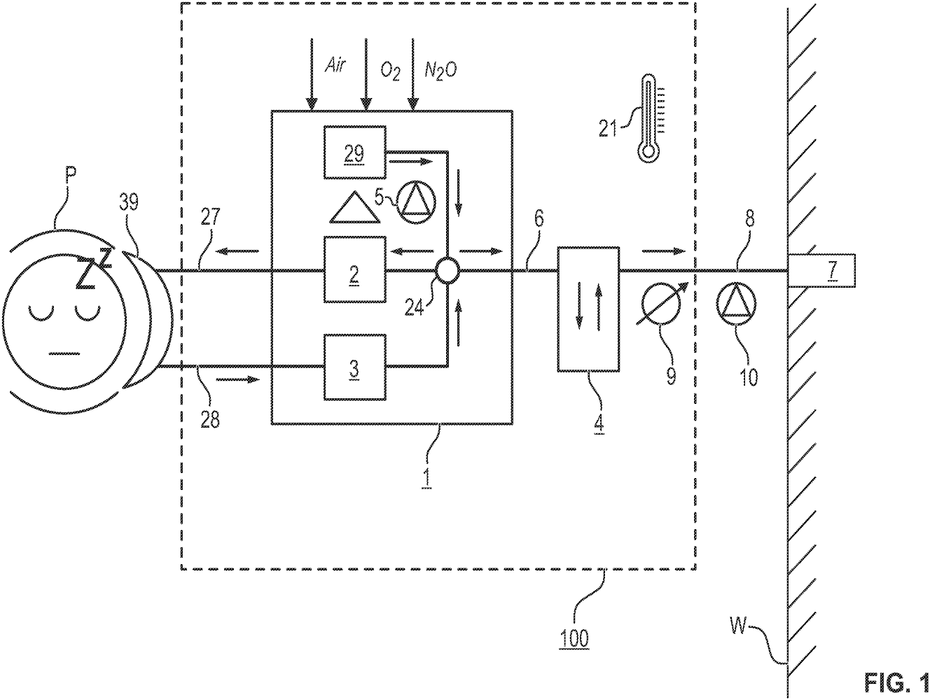

FIG. 1 schematically shows a medical system 100 for anesthetizing or sedating and artificially ventilating the patient P. A schematically shown patient-side coupling unit 39, for example a breathing mask or a tube or a catheter, is positioned on or in the body of the patient P. The patient P is supplied with a gas mixture via an inspiration gas line 27, which flows to the patient-side coupling unit 39. This gas mixture includes oxygen and is mixed with anesthetic to keep the patient P sedated or anesthetized. The percentage of oxygen in the gas mixture may be higher than the percentage of oxygen in the breathing air. The breathable air exhaled by the patient P contains carbon dioxide (CO2) and may also contain traces of the anesthetic administered. The exhaled air is discharged via an expiratory gas line 28, suctioned off in the exemplary embodiment. Both gas lines 27, 28 are connected to a medical device in the form of an anesthesia machine 1 which maintains a flow of gas in a ventilation circuit to supply the patient P with respiratory air and anesthetic and to aspirate and receive exhaled air. This ventilation circuit includes gas lines 27 and 28 and patient-side coupling unit 39 and is passed through the anesthesia machine 1. The two gas lines 27, 28 together form the fluid guide unit of the embodiment, which at least temporarily provides fluid communication between the anesthesia machine 1 and the patient-side coupling unit 39. The gas line 27 serves as the inspiration portion, the gas line 28 as the expiration portion.

The anesthesia machine 1 is supplied with pressurized breathing air, pure oxygen (02) and nitrous oxide (N2O) from a hospital infrastructure and generates the gas mixture. In an embodiment example, the anesthesia machine 1 comprises the following components:
a gas mixer 29 which generates a mixture from at least two of the three supplied gases breathing air, O2 and N20, which mixture is used as a carrier gas for anesthetics, wherein the gas mixer 29 can be constructed as described in DE 10 2008 057 180 B3 (corresponding to US8356596 (B2), which is incorporated by reference),
a fluid delivery unit 5, for example a blower or a pump or a piston-cylinder unit, wherein the fluid delivery unit 5 moves a gas mixture through the ventilation circuit and thereby maintains the gas flow in the ventilation circuit,
an anesthetic vaporizer 2 comprising a tank for liquid anesthetic and a vaporizer unit, and
a preferably device-internal filter unit 3 with a lime filter, the filter unit 3 filtering CO2 out of the respiratory air exhaled by the patient P and discharged via the expiratory gas line 28.

The anesthetic vaporizer 2 adds anesthetic from the anesthetic tank to the carrier gas. For example, the vaporizer unit of the anesthetic vaporizer 2 vaporizes the anesthetic in the tank and/or injects it into the carrier gas.

The anesthesia machine1 supplies gas to the ventilation circuit. The filter unit 3 withdraws gas, in particular CO2, from the ventilation circuit. On balance, more gas is thus supplied to the ventilation circuit than is withdrawn. It is therefore necessary to remove excess gas from the ventilation circuit. This excess gas is hereinafter referred to as “excess gas” and functions as the gas mixture. This excess gas usually contains traces of exhaled anesthetic. The anesthetic is to be filtered out of this gas mixture. In the embodiment example, the anesthetic acts as a gas to be filtered out.

The excess gas is branched off from the ventilation circuit at a branching point 24, by means of a supply line 6 and a subsequent, i.e. downstream, discharge line 8. The branching is effected in two different ways: On the one hand, the fluid conveying unit 5 ejects gas and conveys the ejected excess gas into the feed line 6, wherein the volume flow of the ejected excess gas varies with time and the idealized time course of the volume flow has, for example, the shape of a half-sine curve. On the other hand, the ejected excess gas is passed through the discharge line 8 and, in one embodiment, is sucked in.

In the embodiment, the discharge conduit 8 leads into a stationary fluid receptacle 7 that is embedded in a wall W. The fluid receptacle 7 is preferably part of a stationary infrastructure of a hospital, with the infrastructure receiving gases emitted by various medical devices and passing them on. An intake pump 10 draws gas into the discharge line 8 and conveys it into the fluid receptacle 7. The intake pump 10 may be located in front of or behind the wall W.

An optional volume flow sensor 9 measures the volume flow, i.e. the volume per unit time, flowing through the discharge line 8. For example, the volume flow sensor 9 measures a pressure difference between two measuring points in the discharge line 8, one measuring point being arranged downstream of the other measuring point. In one embodiment, the suction pump 10 is controlled in response to a signal from the volume flow sensor 9 with the control objective that the actual volume flow through the discharge line 8 is equal to a desired predetermined volume flow. Thus, the actual volume flow in the discharge line 8 is automatically controlled.

The feed line 6 directs the excess gas from the anesthesia machine1 to a filter unit 4, which will be described further below and is part of the filter arrangement according to the invention. The excess gas flows at least once through the filter unit 4, optionally several times. Here, the filter unit 4 filters out the anesthetic agent or at least one, preferably each anesthetic agent (the anesthetic) from the excess gas flowing therethrough. The excess gas, cleaned of anesthetic, flows into the discharge line 8.

An ambient temperature sensor 21 measures an indicator of the ambient temperature in the vicinity of the filter unit 4.

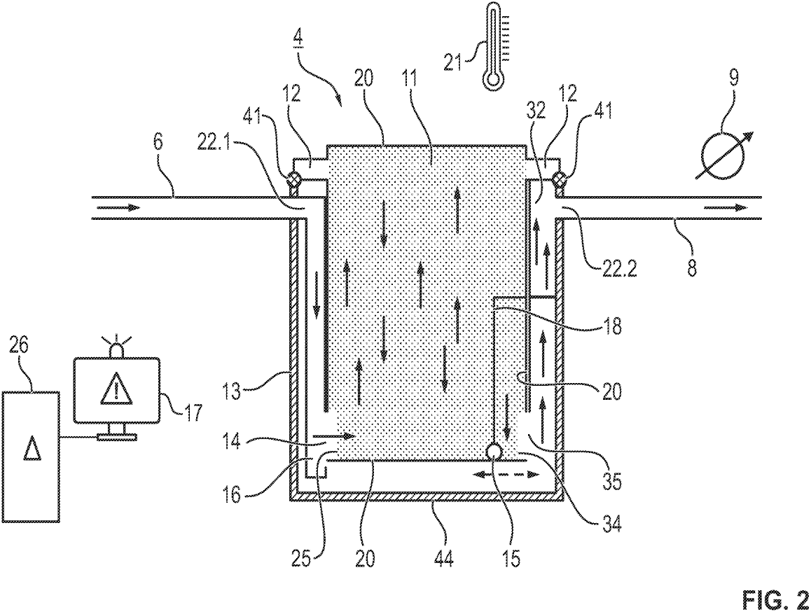

FIG. 2 shows an exemplary embodiment of the filter unit 4. The filter unit 4 comprises a filter element 11 for anesthetics and a cartridge 20 in the form of a cylinder or a truncated cone, the cartridge 20 surrounding the filter element 11. Preferably, the cartridge 20 completely surrounds the filter element 11 in a gas-tight manner except for openings described below. In a preferred embodiment, the filter element 11 comprises a bulk material surrounded and held on all sides by the cartridge 20. Preferably, the bulk material comprises activated carbon. The cartridge 20 prevents bulk material from escaping. It is also possible that the filter element 11 comprises zeolites, organometallic filters and/or silica instead of or in addition to the activated carbon. In the embodiment example, a circumferential protrusion 12 is mounted on the top of the cartridge 20. The filter element 11, the cartridge 20 and the optional protrusion 12 together form the filter of the embodiment.

Furthermore, the filter unit 4 comprises a filter mount in the form of a pot 13, the pot 13 being rotationally symmetrical to a central axis, this central axis being arranged vertically in use and lying in the drawing planes of FIG. 2 and FIG. 5 to FIG. 8. The pot 13 comprises a preferably circular base perpendicular to the central axis and a tubular peripheral surface surrounding the central axis. The pot may also have the shape of a cylinder or a truncated cone. Two openings 22.1 and 22.2 are recessed in the mantle surface of the pot 13, and in the illustrated embodiment near the upper edge of the mantle surface.

A circumferential seal 41 is placed on the upper edge of the circumferential surface of the pot 13. An approximately cylindrical filter 11, 20, 12 can be inserted into this pot 13 from above and removed from the pot 13 again. The circumferential projection 12 is supported on the seal 41 at the upper edge of the circumferential surface. Thanks to the protrusion 12 and the seal 41, the risk of a relevant amount of a gas mixture escaping from the pot 13 into the environment is low. Optionally, a lid not shown can be placed on the pot 13 from above and removed again.

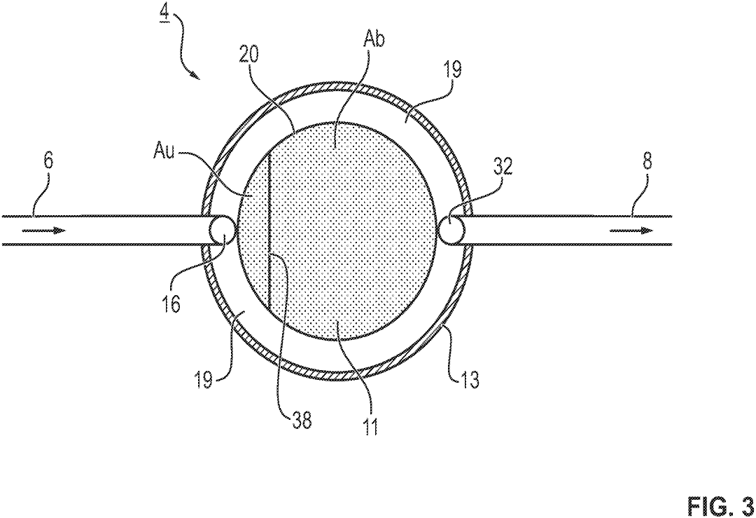

A tubular gap 19 appears between the outer surface of the pot 13 and the cartridge 20, cf. FIG. 3. A pot supply line 16 in the pot 13 is connected in a fluid-tight manner to the supply line 6, conducts excess gas supplied from the opening 22.1 to the bottom of the pot 13 and ends in an outlet opening 14. A pot discharge line 32 in the pot 13 is fluid-tightly connected to the discharge line 8, conducts excess gas exiting the filter 11, 20, 12 towards the discharge line 8, starts in an inlet opening 35 or at the level of the bottom of the cartridge 20 and leads to the opening 22.2.

In one embodiment, when the cartridge 20 is correctly inserted into the pot 13, and in particular in the correct rotational position, the outlet opening 14 of the pot feed line 16 overlaps with an inlet opening 25 in the cartridge 20. The inlet opening 35 of the pot discharge line 32 overlaps with an outlet opening 34 in the cartridge 20. The two openings 14, 35 are located near the lateral surface of the cartridge 20 and near the bottom of the pot 13, and the two openings 25, 34 are located in the lateral surface and near the bottom of the cartridge 20.

In the example shown, the excess gas is introduced into the filter 11, 20, 12 from below through the inlet opening 25. The arrows in the filter element 11 illustrate by way of example the directions in which the excess gas flows through the filter element 11, cf. FIG. 2.

It is also possible that the outlet opening 14 of the pot feed line 16 is located near the lid of the pot 13 and/or the inlet opening 25 in the cartridge 20 is located near the circumferential protrusion 12.

FIG. 3 shows an embodiment in which the excess gas is forced as it flows through the filter unit 11, and therefore flows twice through the filter unit 11. The drawing plane of FIG. 3 is horizontal and perpendicular to the drawing plane of FIG. 2, and the coincident central axis of the pot 13 and the filter 11, 20, 12 is perpendicular to the drawing plane of FIG. 3. It can be seen that the circular gap 19 occurs between the filter 11, 20, 12 and the pot 13.

According to this embodiment, a wall 38 is inserted into the interior of the filter 11, 20, 12, which is impermeable to fluid. This wall 38 extends parallel to the central axis of the filter 11, which is perpendicular to the drawing plane of FIG. 3, preferably at a distance from the central axis, and is preferably in the form of a flat surface or a surface curved along a vertical axis. The wall 38 divides the cartridge 20 and thus the filter element 11 in the cartridge 20 into an ascent region Au and a descent region Ab for the gas mixture flowing therethrough, cf. FIG. 3. Viewed in a viewing direction parallel to the central axis of the filter 11, 20, 12, both the ascent region Au and the descent region Ab each have a cross-section in the form of a segment of a circle. The ascending region Au is in fluid communication with the inlet port 25.

In all embodiments, the filter element 11 filters out anesthetic from the excess gas. The excess gas flows through the supply line 6 into the pot supply line 16 and through the pot supply line 16 and enters the filter element 11 through the inlet port 25 in the cartridge 20. Ideally, all of the anesthetic is removed from the excess gas in the filter element 11. The excess gas then exits the filter element 11 through the outlet opening 34, enters the pot discharge line 32 through the inlet opening 35, and flows through the pot discharge line 32 into the discharge line 8.

The filter element 11 absorbs the anesthetic (the anesthetic comprising one or more anesthetic agents). In many cases, the filter material binds molecules of the anesthetic. This process is exothermic for any filter material used for the filter element 11 of the embodiment. Thus, heat is released when the anesthetic is absorbed. The invention takes advantage of this fact. If activated carbon is used as the filter material, the filter material will in many cases heat up by at least 4° C. at a usual concentration of the anesthetic until the filter element 11 is completely clogged and no further anesthetic can be absorbed. This temperature increase of at least 4° C. can be reliably detected in many cases.

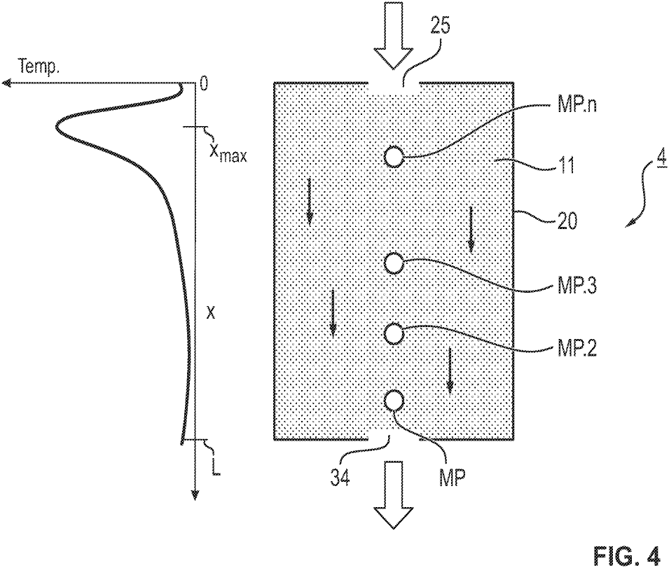

FIG. 4 shows an example of how the temperature inside the filter element 11 at a point in time depends on location, i.e. a spatial pattern. In this schematic diagram, the excess gas flows into the filter element 11 from above through the inlet opening 25, flows through the filter element 11, and exits the filter element 11 through the outlet opening 34, the outlet opening 34 being located at the bottom of the filter element 11. Note: Because the excess gas contains anesthetic, it is generally heavier than air and sinks to the bottom.

The diagram to the left of filter unit 4 shows on the x-axis (from top to bottom) the location x along the direction of flow of the excess gas. L denotes the length of the filter element 11 in the direction of flow, i.e. in FIG. 4 the vertical extent. The location x = 0 stands for the inlet opening 25, the location x = L for the outlet opening 34 of the filter element 11. On the y-axis the location-dependent temperature Temp at a certain point in time is plotted. A maximum at xₘₐₓ can be seen. The filter element 11 is currently taking in anesthetics in a range around this maximum xₘₐₓ. The area from x = 0 to behind x = xₘₐₓ has already largely become clogged with anesthetic.

The temperature peak xₘₐₓ and thus the absorption range in which the filter element 11 currently absorbs anesthetic migrates over time from the inlet opening 25 through the filter element 11 towards the outlet opening 34. A first measuring position MP, at which the current temperature inside the filter element 11 is measured according to the invention, is therefore preferably located in the vicinity of the outlet opening 34. By measuring the temperature at this first measuring position MP, it is possible to detect the event that the temperature peak xₘₐₓ has almost reached the outlet opening 34 and the filter element 11 can only absorb a small amount of further anesthetic.

It is possible that additionally the respective current temperature is measured at further measuring positions MP.2, MP.3, ..., MP.n, wherein these further measuring positions MP.2, MP.3, ..., MP.n are located between the inlet opening 25 and the first measuring position MP. Generally, at a point in time, the respective actual temperature in the filter element 11 differs depending on the measuring position MP, MP.2, ... at which this temperature is measured. The temperature peak xₘₐₓ moves over time from the inlet opening 25 to the outlet opening 34, passing successively through the measuring positions MP.n, ..., MP.2, MP. From the temporal course, i.e. the migration, of the temperature peak xₘₐₓ it is possible in many cases to predict when the filter element 11 will be used up and will therefore have to be replaced.

A signal-processing evaluation unit 26 receives a signal from at least one filter temperature sensor described below and preferably a signal from the ambient temperature sensor 21, cf. FIG. 2 and FIG. 5 to FIG. 7. The measured ambient temperature acts as a reference value and can influence the temperature of the filter element 11. The or each filter temperature sensor respectively measures an indicator of the temperature inside the filter element 11 at a measuring position MP, MP.2, ..., MP.n. The evaluation unit 26 respectively receives a signal from the or each filter temperature sensor and from the ambient temperature sensor 21 and determines a current state of the filter element 11 depending on the or each measured temperature inside the filter element 11 and depending on the measured ambient temperature.

The evaluation unit 26 is able to control a status display 17. If the filter element 11 has absorbed so much anesthetic that the filter 11, 20, 12 needs to be replaced, a message, for example an alarm, is output on the status display 17 in a form that can be perceived by a human being. This message comprises information about the current state of the filter element 11. The alarm may also indicate a period of time after which the filter 11, 20, 12 needs to be replaced.

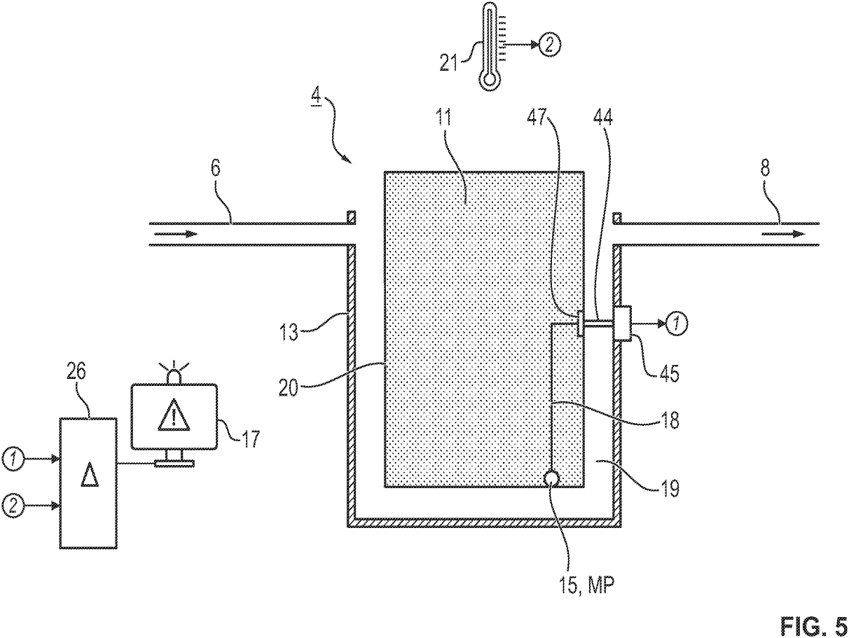

Four possible embodiments of a filter temperature sensor are described below with reference to FIG. 5 to FIG. 8. It is possible that the filter arrangement comprises several filter temperature sensors, whereby several of these configurations are used, which leads to redundancy and in some cases increases the reliability compared to a single measuring principle. It is also possible that the filter arrangement comprises multiple filter temperature sensors configured in the same way, configured to use the same measurement principle.

In the embodiment shown in FIG. 5, a sensing element 15 measures the temperature inside the filter element 11 and there at a first measuring position MP near the outlet opening 34 in the cartridge 20. This sensing element 15 is arranged at the first measuring position MP inside the filter element 11. When the excess gas has reached this first measuring position MP, it has already covered most of its path through the filter element 11. The sensing element 15 is in a data connection with a filter-side contact element 44 inside the pot 13 via a signal line 18, which bridges the gap 19 between the filter 11, 12, 20 and the pot 13. The signal line 18 is arranged inside the filter element 11. A pot side contact element 45 is recessed into the shell surface of the pot 13. The filter-side contact element 44 establishes a data connection between the sensing element 15 and the pot-side contact element 45, and is preferably also mechanically connected to the pot-side contact element 45.

In one embodiment, the cartridge 20 includes a filter-side contact surface 47 that is in electrical contact with the signal line 18 on the inside and in electrical contact with the filter-side contact element 44 on the outside. This filter-side contact surface 47 may comprise a ring or ring segment, so that electrical contact between the signal line 18 and the contact element 44 is established at many or even all possible rotational positions of the filter 11, 20, 12 relative to the pot 13.

The elements 18, 47 and 44 form a data connection between the sensing element 15 and the contact element 45. A signal from the sensing element 15 is transmitted via this data connection to the contact element 45 and from there further to the evaluation unit 26. In addition, the evaluation unit 26 receives measured values from the ambient temperature sensor 21. The evaluation unit 26 compares the signal from the sensing element 15 with the measured ambient temperature and decides whether the filter unit 11 can receive further anesthetic.

It is possible that a further measurement sensor (not shown) is arranged at at least one further measurement position MP.2, ..., MP.n. The measured values of the or each further measuring sensor are also transmitted to the evaluation unit 26 via a signal line and via contact elements.

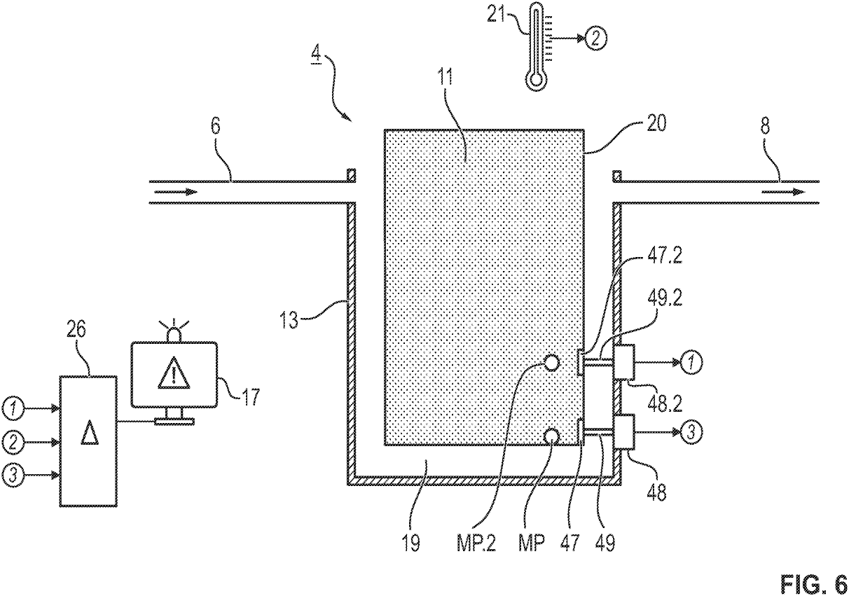

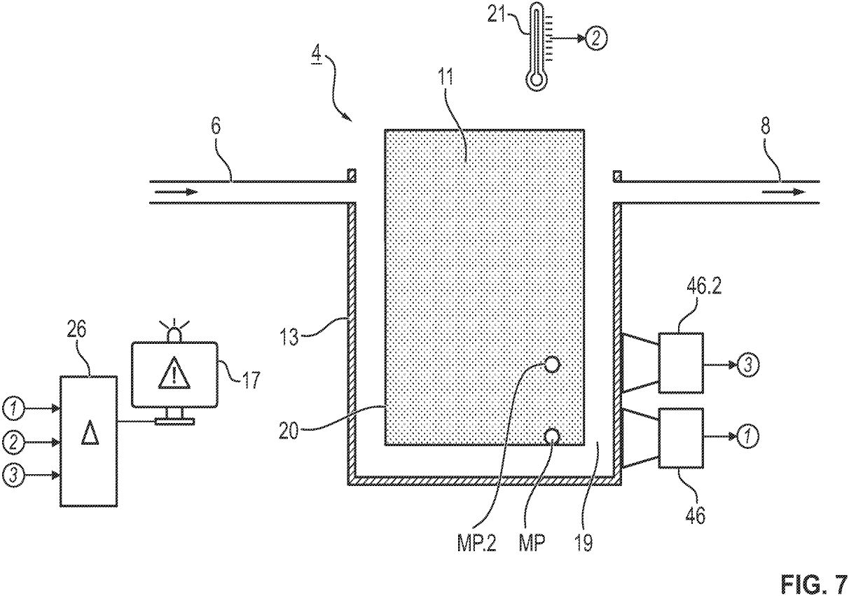

FIG. 6 and FIG. 7 show a preferred embodiment in which it is not necessary to provide the filter element 11 with a sensing element 15 and a signal line 18. Rather, the filter temperature sensor or each filter temperature sensor is located entirely outside of the filter 11, 20, 12, allowing the same filter temperature sensor to be reused sequentially to monitor multiple filters 11, 20, 12. When a spent filter 11, 20, 12 is reprocessed or disposed of, there is no need to give special treatment to a sensing element 15 and a signal line 18.

In the embodiment according to FIG. 6, a filter-side contact element 49 is arranged inside the pot 13 and bridges the gap 19 between the cartridge 20 and the pot 13. The filter-side contact element 49 is in thermal contact with the outer surface of the cartridge 20 and is preferably positioned as close as possible to the measuring position MP. Just as in the embodiment according to FIG. 5, an annular filter-side contact surface 47 may be embedded in the cartridge 20 and as close as possible to the first measuring position MP. However, this filter-side contact surface 47 serves to establish a thermal contact. The elements 47 and 49 together act as a sensing element.

The filter side contact element 49 conducts heat from the filter element 11 to a pot side sensing element 48. The pot-side sensing element 48 acts as a transducer and generates an electrical signal depending on the heat transmitted by the filter-side contact element 49. Examples of implementations of such a sensing element 48 are a thermocouple, a PTC sensor or an NTC sensor.

It is possible that at least one further measuring sensor is positioned in the vicinity of a respective further measuring position MP.2, ..., MP.n. In the example of FIG. 6, a second pot-side sensing element 48.2 is additionally embedded in the outer surface of the pot 13. A second filter-side contact element 49 bridges the gap 19 between the inserted filter 11, 20, 12 and the outer surface of the pot 13. A further filter-side contact surface 47.2 can be inserted into the cartridge 20, namely in the vicinity of the second measuring position MP.2.

FIG. 7 shows an embodiment in which a filter temperature sensor 46 measures an indicator of the temperature occurring at the measurement position MP inside the filter element 11 without contact. This embodiment is less dependent on the relative position of the inserted filter 11, 20, 12 relative to the pot 13 and also less dependent on the actual maximum diameter of the filter 11, 20, 12, which may vary from filter to filter. Preferably, the filter temperature sensor 46 is embedded in the lateral surface of the pot 13 and as close as possible to the measuring position MP. It is possible that at least one further filter temperature sensor 46.2 measures the temperature at a further measuring position MP.2, ..., MP.n without contact.

In a preferred embodiment, the filter temperature sensor 46 measures an indicator of the intensity and/or amount of infrared radiation emanating from the filter element 11 and passing through the cartridge 20 to the outside. The measured values of the filter temperature sensor 46, 46.2 are transmitted to the evaluation unit 26. Examples of a filter temperature sensor 46, 46.2 that measures an indicator of infrared radiation include a pyroelectric sensor, a CCD camera, a thermal imaging camera, multiple infrared thermocouples, or multiple thermopiles. It is also possible to use an infrared camera (thermal imaging camera) as the filter temperature sensor 46.

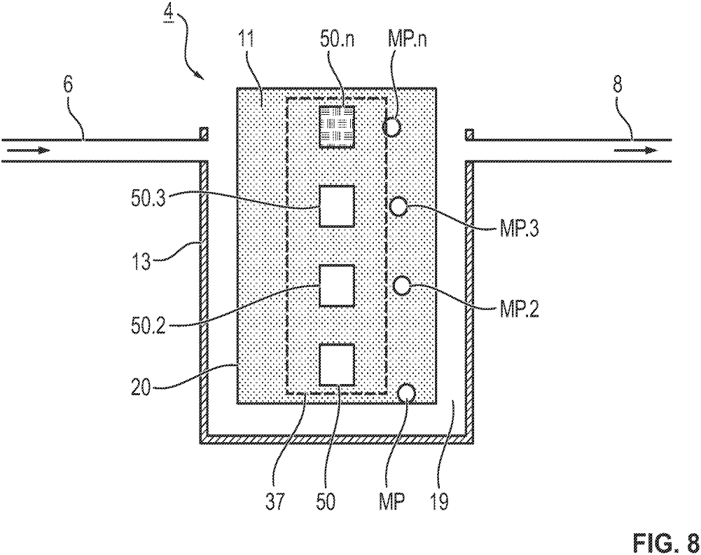

FIG. 8 shows an embodiment which does not require data transmission or a signal-processing evaluation unit 26, but which can be used in combination with such an evaluation unit 26. At least one chemical indicator element 50 is applied to the outer surface of the filter cartridge 20, preferably additionally at least one further chemical indicator element 50.2, ..., 50.n, particularly preferably a sequence of chemical indicator elements 50, 50.2, ..., 50.n, the sequence extending parallel to the central axis of the filter cartridge 20. Each chemical indicator element 50, 50.2, ..., 50.n is located near a respective measurement position MP, MP.2, ..., MP.n. Each chemical indicator element 50, 50.2, ..., 50.n has a first optically perceptible state when the temperature at a measurement position MP, MP.2, ..., MP.n is below a predetermined temperature threshold, and assumes a second optically perceptible state when the cartridge temperature exceeds said temperature threshold, said second state being optically different from said first state. Each sensing position MP, MP.2, ..., MP.n is located on the cartridge 20 and around or in proximity to the respective chemical indicator element. For example, the indicator element 50, 50.2, ..., 50.n changes color when the cartridge temperature exceeds the temperature threshold, thus shows a color change. The temperature threshold is selected such that the cartridge temperature is below this temperature threshold when the filter element 11 has not yet taken up any anesthetic at the respective measuring position MP, MP.2, ..., MP.n, and is above the temperature threshold when the filter element 11 cannot take up any more anesthetic at this measuring position MP, MP.2, ..., MP.n.

In the example of FIG. 8, the indicator element 50.n positioned furthest upstream already has the second state, this state being indicated by hatching, and the remaining indicator elements 50.3, 50.2, 50 still have the first state, which is indicated by white color.

A viewing window 37, shown schematically in FIG. 8, is provided in the peripheral surface of the pot 13. The indicator element or each indicator element 50, 50.2, ..., 50.n is visible from the outside through this viewing window 37. A user can visually detect the current state of the or each indicator element 50, 50.2, ..., 50.n behind the viewing window 37. It is also possible for a camera and an image evaluation unit (both not shown) to automatically determine whether an indicator element 50, 50.2, ..., 50.n is currently in the first state or in the second state. In one embodiment, the camera is recessed in the pot 13 and in another embodiment such that the viewing window 37 is located between the camera and the indicator elements 50, 50.2, ..., 50.n.

While specific embodiments of the invention have been shown and described in detail to illustrate the application of the principles of the invention, it will be understood that the invention may be embodied otherwise without departing from such principles.

List of reference characters:

| | |
|---|---|
| 1 | Anesthesia machine, comprising an anesthetic vaporizer 2, a gas mixer 29, a CO2 lime filter 3, and a fluid delivery unit 5 |
| 2 | Anesthetic vaporizer in anesthesia machine 1, includes anesthetic tank 49 |
| 3 | Lime filter, which filters CO2 out of the exhaled air |
| 4 | Filter unit, which filters out anesthetics from the gas emitted by the anesthesia machine 1, comprises the filter element 11 in the cartridge 20, the cartridge 20 with the projection 12 and the pot 13, is connected to the supply line 6 and the discharge line 8 |
| 5 | Fluid delivery unit of the anesthesia machine 1, which moves gas in the ventilation circuit, is for example in the form of a pump |
| 6 | Feed line, leads from anesthesia machine 1 to filter unit 4 |
| 7 | stationary gas intake of the hospital infrastructure, received by the wall W, connected to the filter unit 4 via the discharge line 8 |
| 8 | Discharge line, leading from the filter unit 4 to the gas intake 7 |
| 9 | Volume flow sensor in the discharge line 8 |
| 10 | suction pump on the discharge line 8 or in the fluid receiver 7, is in fluid communication with the discharge line 8 |
| 11 | cylindrical activated carbon filter of the filter unit 4, acts as a filter element, is surrounded by the cartridge 20 |
| 12 | circumferential projection of the cartridge 20, rests on the upper edge of the pot 13, preferably on the circumferential seal 41 |
| 13 | Pot, into which the feed line 6 leads and from which the discharge line 8 leads, has the shape of a cylinder or truncated cone, acts as a filter mount |
| 14 | Outlet port of pot feed line 16, located at the lower end of pot feed line 16, overlaps with inlet port 25 when cartridge 20 is in place. |
| 15 | Sensing element, which measures the temperature of the filter element 11 at a first measuring position MP inside the filter element 11, is connected to the signal line 18 |
| 16 | Pot supply line inside pot 13, forms a fluid-tight continuation of supply line 6, conducts gas from supply line 6 to the bottom of pot 13, ends in outlet opening 14 |
| 17 | Status display for the filter element 11, is controlled by the evaluation unit 26, can output alarms |
| 18 | Signal line inside the filter element 11, leads from the sensing element 15 to the contact surface 47 |
| 19 | Circumferential gap between the filter 11, 20, 12 and the inner wall of the pot 13 |
| 20 | Cylindrical cartridge, surrounds the filter element 11, comprises the circumferential projection 12, carries in one embodiment the indicator elements 50, 50.2, ... |
| 21 | Ambient temperature sensor, measures an indicator of the temperature in the vicinity of the filter arrangement. |
| 22.1 | Opening in the circumferential surface of the pot 13, in which the supply line 6 ends |
| 22.2 | Opening in the jacket surface of the pot 13, in which the discharge line 8 begins |
| 24 | Branching point in the ventilation circuit at which excess gas is branched off from the ventilation circuit and in which the supply line 6 begins |
| 25 | Inlet opening near the bottom or cover of cartridge 20, overlaps with outlet opening 14 when filter 11, 20, 12 is in place. |
| 26 | Signal processing evaluation unit, which receives measured values from the or each filter temperature sensor and the ambient temperature sensor 21 and determines the current state of the filter element 11. |
| 27 | Inspiratory gas line to supply the patient P with breathing air |
| 28 | Expiratory gas line to aspirate breathing air exhaled by patient P |
| 29 | Gas mixer of the anesthesia machine 1, generates the carrier gas for the anesthetic |
| 32 | Pot discharge line inside pot 13, starts in inlet opening 35, directs gas from bottom of pot 13 into discharge line 8 |
| 34 | Outlet opening near the bottom of cartridge 20, overlaps with inlet opening 35 when filter 11, 20, 12 is in place. |
| 35 | Inlet port of pot discharge line 32, is in fluid communication with outlet port 34 when filter 11, 20, 12 is in place. |
| 37 | Inspection window in the jacket surface of the pot 13 |
| 39 | Patient-side coupling unit, positioned in or on the patient’s body P |
| 41 | Circumferential seal on the upper rim of the pot 13 |
| 44 | Contact element on the filter side, which electrically bridges the gap 19 between the pot 13 and the filter 11, 20, 12 |
| 45 | Pot-side contact element in the wall of the pot 13, comes into thermal contact with the filter-side contact element 44, generates measured values and forwards these to the evaluation unit 26 |
| 46 | Non-contact filter temperature sensor, measures an indicator of the infrared radiation generated by the filter element 11 at the measuring position MP |
| 46.2 | Second non-contact filter temperature sensor, measures an indicator of the infrared radiation generated by the filter element 11 at the measuring position MP.2 |
| 47 | Contact surface in the cartridge 20 and in the vicinity of the first measuring position MP, comes into contact with the signal line 18 (only in the embodiment with the sensing element 15) and the filter-side contact element 44, 49 |
| 47.2 | Contact surface in the cartridge 20 and near the first measuring position MP, comes into contact with the filter-side contact element 49.2 |
| 48 | Pot-side sensing element in the outer surface of the pot 13, is in thermal contact with the filter-side contact element 49, forwards measured values to the evaluation unit 26 |
| 48.2 | Further sensing element on the pot side in the outer surface of the pot 13, is in thermal contact with the contact element 49.2 on the filter side, forwards measured values to the evaluation unit 26 |
| 49 | Contact element inside the pot 13, comes into contact with the contact surface 47, thermally bridges the gap 19 between the pot 13 and the cartridge 20, |
| 49.2 | Further contact element inside the pot 13, comes into contact with the contact surface 47.2, thermally bridges the gap 19 between the pot 13 and the cartridge 20, |
| 50 | Chemical indicator element in the vicinity of the first measuring position MP, has a first state (low temperature) or a second state (high temperature) depending on the temperature of the filter element 11 |
| 50.2, ..., 50.n | Further chemical indicator elements near the measuring positions MP.2, ..., MP.n |
| 100 | Medical system for artificial ventilation of patient P, comprising the anesthesia machine 1, the filter unit 4, the lines 6 and 8, the volume flow sensor 9 and the ambient temperature sensor 21, can be connected to the coupling unit 39 on the patient side and the gas intake 7 |
| L | Length of the filter element 11 |
| MP | First measuring position, at which the temperature in the filter element 11 is measured, is located close to the outlet opening 34 |
| MP.2, ..., MP.n | Further measuring positions inside the filter element 11, arranged upstream of the first measuring position MP |
| P | Patient connected to the anesthesia machine 1 via the patient-side coupling unit 39 and breathing at least one anesthetic (anesthetic) |
| Temp | Temperature inside the filter unit 11 |
| W | Wall receiving the stationary gas intake 7 |
| Xmax | Position of the temperature peak, i.e. position of the maximum temperature inside the filter element 11 |

What is claimed is:

1. A filter arrangement for filtering out a gas from a gas mixture, the filter arrangement comprising:
a filter unit comprising an inlet and an outlet,
wherein the filter unit is configured to filter gas out of the gas mixture with the gas mixture flowing through the filter unit, the filter unit is configured to take up the gas, wherein the filter unit heats up as a consequence of taking up the gas,
wherein the filter arrangement is configured such that the gas mixture flows through the inlet into the filter unit, flows at least once through the filter unit and flows through the outlet out of the filter unit;
a sensor arrangement comprising a filter temperature sensor configured to measure at least once an indicator of a temperature in a measuring area inside the filter unit,
wherein the filter arrangement is configured:
to generate a message depending on the measured temperature; and
to output the message or cause that message to be output in a form that is perceptible by a human being, and
wherein the message comprises information about a current state of the filter unit.

2. A filter arrangement according to claim 1, further comprising a signal-processing evaluation unit configured:
to decide, using a signal from the filter temperature sensor, whether a predetermined criterion is met, wherein the criterion depends on at least one value of the temperature in the first measuring area; and
to generate the message with the information about the state of the filter unit if the criterion is met.

3. A filter arrangement according to claim 2, wherein:
the filter temperature sensor is configured to measure the indicator of temperature in the measuring area at a plurality of successive sampling times;
the signal-processing evaluation unit is configured to determine a time course of the temperature in the measuring area using a signal from the first filter temperature sensor, and
the criterion for which the message is generated when the criterion is met depends on the time course of the temperature in the measuring area.

4. A filter arrangement according to claim 2, wherein:
the filter temperature sensor is a first filter temperature sensor and the measuring area is a first measuring area;
the sensor arrangement further comprises a second filter temperature sensor configured to measure at least once an indicator of a temperature in a second measuring area inside the filter unit;
the first measuring area, with respect to a direction in which the gas mixture flows through the filter unit, is arranged downstream of the second measuring area;
the signal-processing evaluation unit is configured to use a signal from the first filter temperature sensor and a signal from the second filter temperature sensor to determine a spatial course at a point in time of the temperature along a distance from the inlet to the outlet of the filter unit; and
the criterion for which the message is generated depends on the detected spatial course of the temperature along the distance.

5. A filter arrangement according to claim 2,
wherein the sensor arrangement comprises an ambient temperature sensor configured to measure an indicator of a temperature in the environment of the filter assembly as a measured ambient temperature or is adapted to receive a signal containing a measured ambient temperature from an ambient temperature sensor,
wherein the evaluation unit is configured to calculate a difference between a filter temperature measured by the filter temperature sensor and the measured ambient temperature,
wherein the criterion depends on the difference between the measured filter temperature and the measured ambient temperature.

6. A filter arrangement according to claim 1, wherein:
the filter unit comprises a filter mount and a filter;
the filter is inserted or insertable into the filter mount;
the filter arrangement is configured such that with the filter inserted into the filter mount, the gas mixture flows into the filter mount, through the inlet, through the filter, out of the outlet and out of the filter mount; and
the filter temperature sensor is inserted into a wall of the filter mount and is spaced from the inserted filter.

7. A filter arrangement according to claim 6, wherein the filter temperature sensor comprises an infrared sensor configured to measure an indicator of an amount or of an intensity of infrared radiation emitted by the filter, as the indicator of temperature.

8. A filter arrangement according to claim 7, wherein the infrared sensor comprises one or more of:
a pyroelectric sensor;
a thermal imaging camera;
several infrared thermocouples; and
several thermo-piles.

9. A filter arrangement according to claim 6, wherein:
the filter temperature sensor comprises a thermal probe and a transducer;
the thermal probe establishes thermal contact between the inserted filter and the transducer; and
the transducer is configured to generate a signal dependent on a temperature of the thermal probe.

10. A filter arrangement according to claim 6, wherein the filter temperature sensor comprises:
a sensing element inside the filter, wherein the sensing element is configured to generate a signal depending on the temperature in the measuring area; and
a receiver in the wall of the filter mount, wherein with the filter inserted into the filter mount, a data link is established between the sensing element and the receiver.

11. A filter arrangement according to claim 1, wherein:
the filter temperature sensor comprises a chemical indicator element being in thermal contact with the filter and having dependent on the temperature of the filter in the measuring area a first optically perceptible state or a second optically perceptible state; and
the second optically perceptible state is different from the first optically perceptible state.

12. A filter arrangement according to claim 11, wherein
the filter unit comprises a filter mount and a filter;
the filter is inserted or insertable into the filter mount;
the filter arrangement is configured such that with the filter inserted into the filter mount, the gas mixture flows into the filter mount, through the inlet, through the filter, out of the outlet and out of the filter mount;
wherein the chemical indicator is mounted onto the filter; and
wherein a viewing window is inserted into the filter mount such that the chemical indicator is visible from outside through the viewing window with the filter being inserted into the filter mount.

13. A filter arrangement according to claim 1, wherein the filter unit is configured to filter out anesthetic from the gas mixture.

14. A ventilation system for ventilation of a patient, the ventilation system comprising:
a ventilator;
a fluid guide unit comprising an inspiration portion, the fluid guide unit being configured to at least temporarily establish a fluid connection between the ventilator and a patient-side coupling unit, wherein the patient-side coupling unit is connectable to a patient, wherein the ventilator is configured to deliver a gas mixture comprising oxygen through the inspiration portion to the patient-side coupling unit; and
a filter arrangement at least temporarily in fluid communication with the fluid guide unit and configured to filter out gas from a gas mixture, the filter arrangement comprising:
a filter unit comprising an inlet and an outlet, wherein the filter unit is configured to take up the gas, wherein the filter unit heats up as a consequence of taking up the gas, wherein the filter arrangement is configured such that the gas mixture flows through the inlet into the filter unit, flows at least once through the filter unit and flows through the outlet out of the filter unit;
a sensor arrangement comprising a filter temperature sensor configured to measure at least once an indicator of temperature in a measuring area inside the filter unit, wherein the filter arrangement is configured: to generate a message depending on the measured temperature; and to output the message or to cause that message to be output in a form that is perceptible by a human being, and wherein the message comprises information about a current state of the filter unit.

15. A ventilation system according to claim 14, wherein:
the ventilator is configured as an anesthesia machine; and
the fluid guide unit comprises an expiration portion and is configured to establish a ventilation circuit between the anesthesia machine and the patient-side coupling unit,
the anesthesia machine is configured to convey a gas mixture comprising oxygen and at least one anesthetic to the patient-side coupling unit through the inspiration portion, and
wherein the filter arrangement is at least temporarily in a fluid connection with the expiration portion and is adapted to filter out the anesthetic from the gas mixture which is passed through the filter unit of the filter arrangement.

16. A process for filtering a gas from a gas mixture using a filter arrangement which comprises a filter unit and a sensor arrangement with a filter temperature sensor, wherein the filter unit comprises an inlet and an outlet, the process comprising the steps of:
providing a gas mixture such that the gas mixture flows through the inlet into the filter unit with the gas mixture flowing through the filter unit at least once and flows through the outlet out of the filter unit;
filtering gas out of the gas mixture with the filter unit while the gas mixture flows through the filter unit;
taking up the filtered-out gas with the filter unit heating up as a result of taking up of the gas;
with the filter temperature sensor, measuring an indicator of a temperature in a measuring area inside the filter unit;
generating a message depending on the measured temperature; and
outputting the generated message or causing the generated message to be output in a form that can be perceived by a human being, said message comprising information about a current state of the filter unit.

17. A process according to claim 16, wherein the process further comprises:
deciding whether a predetermined criterion is fulfilled, the criterion depending on the temperature in the first measuring area; and
generating and outputting the message with the information about the state of the filter unit when the criterion is fulfilled.

18. A process according to claim 16, wherein the process comprises the further steps of:
with the filter temperature sensor, measuring the indicator of temperature in a measurement region inside the filter unit at a plurality of successive sampling times;
determining a time course of the measured temperature in the first measuring area; and
providing the criterion for generating the message so that the criterion depends on the time course of the temperature in the first measuring area.
